(19) United States
(12) Patent Application Publication
(10) Pub. No.: US 2010/0255045 A1
(43) Pub. Date: Oct. 7, 2010

(54) COMPOSITION FOR PHOTODYNAMIC SKIN TREATMENT

(76) Inventor: Michèle Eymard Du Vernet, Paris (FR)

Correspondence Address: SOFER & HAROUN LLP., 317 MADISON AVENUE, SUITE 910, NEW YORK, NY, 10017, US

(21) Appl. No.: 12/744,399

(22) PCT Filed: Nov. 27, 2008
(86) PCT No.: PCT/FR08/01650

§ 371 Date: May 24, 2010

(30) Foreign Application Priority Data
Nov. 27, 2007 (FR) 0708284

(51) Int. Cl. A61K 8/02; C07F 1/08; C07F 15/06; C07F 15/04; C07F 13/00; C07F 3/06; C07F 15/02; A61K 9/00; A61K 31/555; A61K 31/58; A61K 8/58; A61K 8/63; A61Q 19/08; A61Q 19/02; A61P 17/10; A61P 17/06; A61K 31/60

(52) U.S. Cl. 424/401; 540/145; 424/484; 514/185; 514/171; 514/161

(57) ABSTRACT

The invention relates to a composition for photodynamic therapy. The composition of the invention, in the form of a gel or a cross-linked hydrogel, includes a chlorophyll photosensitising substance or a derivative thereof, in particular chlorophyllin optionally associated with an additional ingredient such as an alpha- or beta-hydroxy acid, a depigmentation or lightening agent, and/or an antioxidant. The invention can be used in the treatment of skin aging signs and in skin regeneration.

The present invention relates to a novel cosmetic and dermatological composition that can be used in a photodynamic technique, and more particularly a composition based on chlorophyll and chlorophyllin, and derivatives thereof, that can be used for skincare by means of the technique used in photodynamic therapy, and in particular for the prevention and treatment of skin aging signs.

The skin constitutes, on its own, an organ which performs several functions that are determining factors for the health of human beings or animals. It constitutes a barrier with respect to the environment, and the quality of its defenses with respect to attacks from the environment is therefore a major element in terms of the effectiveness of its protection.

The skin comprises several integrated layers, namely a superficial layer constituted of the epidermis, and two deeper layers forming the dermis and the hypodermis. Each of these layers has specific properties enabling the whole to react and adapt to the conditions of its environment.

The epidermis is mainly composed of keratinocytes (90% of epidermal cells), melanocytes (2 to 3% of epidermal cells) and Langerhans cells. The thickness of the epidermis is variable depending on the various parts of the body. The dermis is thicker, and is composed mainly of collagen, elastin and proteoglycans. These three types of molecules are synthesized by the dermal fibroblasts. The collagen fibers provide the mechanical strength and the texture of the skin, the elastin is responsible for the elasticity, and the proteoglycans play a major role in terms of the structure and hydration of the skin. Other cells, such as macrophages and leucocytes, are also present in the dermal layer.

The hypodermis is the deepest layer of the skin, and contains the adipocytes which produce lipids so that the subcutaneous tissue can produce a fatty layer that protects the muscles, bones and internal organs against impacts.

The skin immune system groups together a large number of cell participants, such as keratinocytes, Langerhans cells, fibroblasts, mast cells, macrophages and endothelial cells, and also leucocytes, and various soluble inflammatory mediators such as cytokines and chemokines. This skin immune system ensures maintenance of the homeostasis of the skin. It is also responsible for the activation and regulation of normal and pathological inflammatory reactions.

Skin aging can be intrinsic, or extrinsic, i.e. caused by the environment, including climatic attacks, which can in particular contribute to accelerating collagen degradation in the dermis, and in particular exposure to sunlight, temperature variations and free radicals. The first signs of skin aging, such as wrinkles and fine lines, are generally caused by stress and biological and physiological changes, accelerated by the external environment or by ways of life. The appearance of pigmentary marks, a decrease in thickness of the skin and sagging thereof are also changes that are observed during aging. It is known that the ability of the skin to replace damaged collagen decreases over time, and, consequently, spaces and irregularities appear in the collagen network.

Various treatments have been envisioned in an attempt to limit skin degradation under the effect of aging, or even to cause rejuvenation of the skin cells so as to improve the appearance and the condition of the skin.

Thus, treatments by means of compositions in the form of sera, creams and lotions containing alpha-hydroxy acids or retinoids, applied regularly in order to gradually reduce the number of wrinkles and fine lines, or else topical compositions based on pentapeptides of the Lys-Thr-Thr-Lys-Ser type, promoting collagen and glycosaminoglycan synthesis and providing an effect on skin generation, as described in patent FR-A-2.783.169, have been proposed. Collagen and hyaluronic acid implants for concealing expression lines around the eyes or mouth, dermabrasion and chemical peels for removing the upper layer of damaged skin, esthetic surgery, such as blepharoplasty (eyelid surgery) or a lift for tautening skin showing sagging, or else restructuring using a carbon dioxide laser for eliminating wrinkles, have also been proposed.

Studies have shown that treatments using the photodynamic therapy (PDT) technique can also be used. Specifically, it is known that certain substances have photosensitizing properties and can be used in photodynamic therapy for the treatment and diagnosis of certain diseases. For example, photodynamic therapy techniques have been proposed in dermatology for the treatment of actinic keratoses, psoriasis and vitiligo, in opthalmology for the treatment of age-related macular degeneration, and also in cancerology for the treatment of brain tumors, tumors of the digestive tract, lung tumors or prostate tumors.

The PDT technique consists in administering the photosensitizing substance, which preferentially binds in the tissues, for example to the tumor cells, and in subsequently irradiating the tissues by means of a light source of appropriate wavelength, capable of activating the photosensitizing substance. The photosensitizing substance, activated by a monochromatic light (laser or electroluminescent diode) or polychromatic light (lamp), goes from its initial base-energy state to a higher energy state, and can release, in situ, singlet oxygen or free radicals which are extremely reactive and oxidize the immediately surrounding tissues, causing death of the cells, and in particular of the cancer cells. These techniques are described, in particular, by K. R. Weishaupt et al., Cancer Research, p. 2326-2329 (1976) and T. J. Dougherty et al. “Photodynamic therapy (PDT) and clinical applications”, Biomedical Photonics Handbook. CRC Press LLC. 38: 1-38 (2003). The oxygen free radical species thus produced generally diffuse weakly and have a very short lifetime, with the result that their toxic effect is very localized.

The first substances studied in this field are hematoporphyrin derivatives such as porfimer sodium (Photofrin®), which is a mixture of dihematoporphyrin esters and ethers. However, Photofrin® has the drawback of having to be administered intravenously two days before the light-source activation treatment, and its accumulation in the organism before elimination causes skin photosensitization for 4 to 8 weeks; furthermore, at the wavelength used (630 nm), light penetration is limited to a few millimeters, and the use of this photosensitizing agent is therefore limited to small tumors located at the surface of tissues.

Many other derivatives have been proposed for treatments by PDT, for example benzoporphyrin derivatives, which have the advantage of being activated at higher wavelengths than Photofrin (approximately 690 nm), thereby resulting in better penetration of the light rays into the tissues. In addition, benzoporphyrin derivatives are eliminated more rapidly from the organism and have fewer side effects than Photofrin®, in particular a weaker skin photosensitization. A benzoporphyrin-based medicament, verteporfin (Visudyne®), is used for the treatment of age-related macular degeneration. Patent EP 1834955 describes porphyrin derivatives that can be used in anticancer PDT as photosensitizers.

It has also been proposed to use the photosensitizing effect of aminolevulinic acid (ALA) for the treatment of epidermal cells, as indicated by J. S. Dover et al., Archives of Dermatology, vol. 141, p. 1247-1252 (October 2005). Aminolevulinic acid acts as a precursor of protoporphyrin IX, and has the effect, after activation by light at a wavelength of approximately 630 nm, of destroying epidermal cells and causing a subsequent photorejuvenating effect. The methyl ester of aminolevulinic acid (Metvix®) has a better bioavailability and is proposed in the treatment of actinic keratoses and superficial carcinomas. However, it is known that the degree of penetration of aminolevulinic acid through the skin is relatively low, even in ester form, which limits its bioavailability. In addition, the use of aminolevulinic acid can lead to the appearance of erythemas depending on the doses used.

More recently, it has been shown that bacteriochlorophyll derivatives, for instance those described in patent EP 584552, have advantageous photosensitizing properties that can be used in photodynamic therapy.

The use of compounds with a porphin ring, extracted from chlorophyllous plants, such as chlorophyll and pheophytin, in cosmetic compositions having skin protecting and restructuring effects, has been described in patent application WO 9402484. Other bacteriochlorophyll derivatives that can be used in anticancer therapy by PDT are described in patent application WO 0140232.

However, no photosensitizing substance that can be used in dermatology by means of the photodynamic therapy technique in order to effectively treat skin aging signs is known.

It is therefore desirable to be able to have novel compositions suitable for the dermatological or cosmetic treatment of the skin by means of the photodynamic therapy (PDT) technique.

The studies carried out by the applicant have shown that it is possible to use chlorophyll and some of its derivatives effectively, as a photosensitizing substance for treatments by PDT, in order to treat skin aging signs or to cause rejuvenation of the skin, and that this photosensitizing substance can be advantageously combined, within the same composition, with certain other active ingredients which have a complementary activity.

The studies carried out by the applicant have also shown that certain chlorophyll derivatives, in particular chlorophyllin, play a protective role limiting the side effects of aminolevulinic acid, preventing in particular the appearance of erythema, and promoting the effectiveness of said aminolevulinic acid.

A subject of the present invention is therefore a novel composition based on chlorophyll or on a derivative thereof, that is of use in cosmetics and in dermatology for the treatment of skin aging signs by photodynamic therapy.

A subject of the present invention is also a cosmetic and/or dermatological composition based on chlorophyll or on a derivative thereof, for treating the skin by rejuvenating the skin cells by means of the photodynamic therapy technique.

A subject of the invention is also a cosmetic and/or dermatological composition combining chlorophyll or a derivative thereof with aminolevulinic acid (ALA) and/or an alpha-hydroxy acid and/or a depigmenting agent.

A subject of the invention is also a method for cosmetic treatment of the skin by means of a composition based on chlorophyll or on a derivative thereof, suitable for the photodynamic therapy technique.

Finally, a subject of the present invention is also the use of a composition based on chlorophyll or on a derivative thereof, for the preparation of a medicament for preventing and treating skin aging signs and rejuvenating the skin.

According to one principal characteristic of the present invention, the composition contains chlorophyll or a chlorophyll derivative such as chlorophyllin, as photosensitizing substance.

Chlorophyll is a pigment that is found in many plants, in particular in the chloroplasts of plant cells.

In the present invention, use may be made of the various chlorophylls a, b, c and d, and also chlorophyllin and their derivatives obtained by hydrolysis.

Chlorophyll and its derivatives that can be used in the invention can be represented by general formula (I) below:

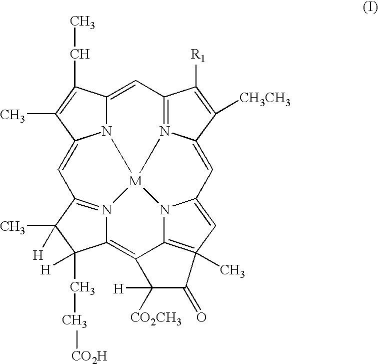

in which M represents a metal atom and R₁ represents a lower alkyl group containing from 1 to 4 carbon atoms, and preferably a methyl group —CH₃ (corresponding to chlorophyll a) or a formyl group —CHO (corresponding to chlorophyll b).

The metal atom M is chosen from magnesium, copper, cobalt, nickel, manganese, zinc and iron. Chlorophyll is represented by formula (I) above where M is a magnesium atom. The chlorophyllin used in the present invention is represented by the same formula (I) above where M is a metal other than magnesium. Chlorophyllin differs in particular from chlorophyll by virtue of the fact that it has good water-solubility, while at the same time conserving substantially the same photosensitizing effect.

According to one variant, in addition to the chlorophyll derivative, the composition of the invention comprises aminolevulinic acid, and, according to one preferential embodiment, it comprises a combination of chlorophyllin and aminolevulinic acid. The studies carried out have in fact shown a faster and stronger action with this combination compared with the use of chlorophyllin on its own.

The composition according to the invention is preferably provided in the form of a crosslinked hydrogel or gel, which allows gradual release of the active ingredients and provides good homogenization of the penetration into the epidermis. The crosslinked hydrogel makes it possible to increase transdermal penetration of the photosensitizing compound, while at the same time controlling its gradual release from the hydrogel applied to the skin. The hydrogel is particularly suitable for the combination of chlorophyllin and aminolevulinic acid, of which the degree of penetration through the skin is then improved. It is also possible to use known encapsulation or vectorization techniques for providing the regulated release of the active ingredients.

This composition can be applied to the skin and is intended to be irradiated by a light source of wavelength between 610 and 650 nm, preferably between 625 and 635 nm, causing activation of the photosensitizing compound (chlorophyll, chlorophyllin and derivatives thereof). The use of a light source at this wavelength makes it possible to act with satisfactory penetration of the photosensitizing agent, of the order of from 5 to 8 mm approximately, i.e. at the level of the dermis.

This treatment is preferably carried out after having carried out a first peel by means of a composition based on an alpha-hydroxy or beta-hydroxy acid, for example glycolic acid, lactic acid, malic acid, tartaric acid, citric acid, or salicylic acid, and preferably glycolic acid as alpha-hydroxy acid and salicylic acid as beta-hydroxy acid. The treatment can also be carried out simultaneously with the peel.

According to one advantageous embodiment of the invention, the composition comprises an alpha-hydroxy or beta-hydroxy acid, in addition to the photosensitizing compound. It is thus possible to carry out a first peel treatment by applying the composition to the skin while the photosensitizing compound gradually migrates into the epidermis and the dermis, and then the photosensitizing compound is activated by exposing the skin to light of suitable wavelength.

According to one variant embodiment of the invention, it may be advantageous to incorporate into the composition one or more compounds having a complementary activity, and, for example, a depigmenting agent such as arbutin, glabridin, vitamin C (ascorbic acid) or kojic acid. The tests carried out have shown that the depigmenting effect is potentiated by the use of the composition of the invention in the context of a photodynamic therapy technique. The treatment may then be particularly effective against strong pigmentation marks resulting, for example, from a dermatological laser treatment.

According to one advantageous embodiment, the composition based on chlorophyll or on chlorophyllin is combined with a depigmenting agent, such as kojic acid or an ester thereof, such as the dipalmitate, arbutin or a derivative thereof, or else glabridin.

It may also be advantageous to incorporate a complementary active agent such as diosgenin, or a diosgenin ester, used for its adaptogenic action promoting induction of the cell metabolism so as to reestablish homeostasis, and for its free-radical-scavenging capacity with respect to superoxide action, making it possible to control the level of free radicals induced by the action of the PDT.

A complementary active ingredient that can be used in the compositions of the invention can also be a powerful antioxidant such as superoxide dismutase, phytic acid, coenzyme Q10, or diosgenin. It is also advantageous to add a hydrolyzed glycosaminoglycan to the composition.

According to one variant embodiment of the invention, it is possible to envision two compositions applied successively, the first containing a complementary active ingredient, while the second contains the photosensitizing compound.

As indicated above, the photosensitizing compound used in the invention is chlorophyll, in particular chlorophyll a or chlorophyll b, or preferably chloro-phyllin, and more particularly 5-CR-chlorophyllin®.

The treatment by means of the composition of the invention has the advantage of stimulating fibroblast activity and collagen and elastin synthesis in the dermis, thus leading to restructuring of the skin that is in general visible from the 3rd or 4th treatment session onward. The duration of each treatment session depends on the diagnosis by the practitioner and on the condition of the patient, and, in general, it can be between approximately 10 and 30 minutes.

The skin rejuvenating effect results from the action of the free radicals originating from the photo-sensitization, which cause an inflammatory reaction and a tissue healing process without burning. It manifests itself through a contraction of the skin, which leads to a decrease in wrinkles and fine lines, and improves the oval of the face.

The tests carried out with compositions in accordance with the present invention have demonstrated an increase in volemia, i.e. in total blood volume, and in particular in the microcirculation, and in the level of oxygen in the epidermal tissue.

Depending on the complementary active ingredients, the compositions according to the invention can be used effectively for treating dermatological conditions such as psoriasis, acne, vitiligo or melasma, with no recurrence in the latter case, both on light skin and on dark skin.

The following examples illustrate the invention without limiting the scope thereof. In these examples, the parts and percentages are given by weight, unless otherwise indicated.

### EXAMPLE 1

The photosensitizing formulations for treatment by PDT, indicated hereinafter, are prepared by usual methods.

### Formulation A

| | | |
|---|---|---|
| Xanthan gum | | 0.5 |
| Chitosan | | 0.4 |
| Chlorophyllin | | 10.0 |
| Diosgenin | | 1.0 |
| Preservative | | 1.0 |
| Water | qs | 100.0 |

### Formulation B

| | | |
|---|---|---|
| Xanthan gum | | 0.5 |
| Chitosan | | 0.4 |
| Chlorophyllin | | 5.0 |
| Salicylic acid | | 10.0 |
| Diosgenin | | 1.0 |
| Preservative | | 1.0 |
| Water | qs | 100.0 |

### Formulation C

| | | |
|---|---|---|
| Xanthan gum | | 0.5 |
| Chitosan | | 0.4 |
| Chlorophyllin | | 10.0 |
| Salicylic acid | | 10.0 |
| Kojic acid | | 5.0 |
| Phytic acid | | 3.0 |
| Preservative | | 1.0 |
| Water | qs | 100.0 |

### Formulation D

| Glycolic acid | | 1.0 |
|---|---|---|
| Poly(methyl methacrylate) | | 2.5 |
| Propylene glycol dicaprylate/dicaprate | | 3.5 |
| Glyceryl stearate | | 1.0 |
| Oleic acid | | 0.8 |
| Polysorbate 60 | | 1.0 |
| PEG-100 stearate | | 0.8 |
| Ubiquinone | | 0.8 |
| Xanthan gum | | 0.5 |
| Chlorophyllin | | 2.5 |
| Preservative | | 0.5 |
| Water | qs | 100.0 |

### Formulation E

| | | |
|---|---|---|
| Aminolevulinic acid | | 1.0 |
| Chlorophyllin | | 8.0 |
| Xanthan gum | | 0.5 |
| Chitosan | | 0.5 |
| Diosgenin | | 1.0 |
| Preservative | | 0.5 |
| Water | qs | 100.0 |

### EXAMPLE 2

### Clinical Study

A study was carried out on 20 women over the age of 50, with pronounced dermatitis solaris and actinic keratoses or wrinkles on the face and neck.

The total duration of the study was 9 weeks, consisting in applying the treatment by PDT with the composition in accordance with formulation D of example 1, exposing the areas to be treated for 20 minutes (wavelength used: 633 nm).

Each application of the composition is carried out in the following way.

The area to be treated (face and, optionally, neck) is cleaned using a nonalcoholic product and rinsed with water and then dried. The composition of the invention is then applied (crosslinked hydrogel and activator) and left on for approximately 20 minutes.

The irradiation (Omnilux Revive lamp, 633 nm) is then carried out for 20 minutes, with the patient's eyes being protected with a suitable mask.

A soothing cream is then applied to the entire area treated.

The study of the treated cases showed a clear improvement in the condition of each patient, from the 3rd application onward, without any scarring after-effects.

1. A cosmetic and/or dermatological composition for treating the skin by rejuvenating the skin cells by means of the photodynamic therapy technique comprising as photosensitizing substance:
chlorophyll or a derivative thereof, represented by general formula (I): 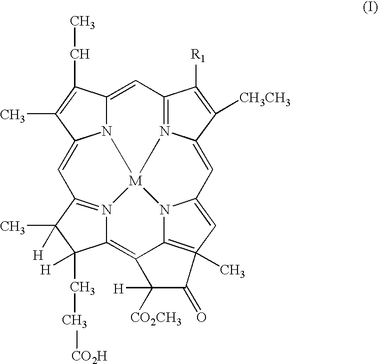
in which M represents a metal atom and R₁ represents a lower alkyl group containing from 1 to 4 carbon atoms.

2. The composition as claimed in claim 1, wherein in general formula (I), R₁ represents a methyl group —CH₃ (corresponding to chlorophyll a) or a formyl group.

3. The composition as claimed in claim 2, wherein M is a metal atom chosen from magnesium, copper, cobalt, nickel, manganese, zinc and iron.

4. The composition as claimed in claim 1, wherein the photosensitizing substance is chosen from the group consisting of chlorophyll a, chlorophyll b, chlorophyllin and their derivatives obtained by hydrolysis.

5. The composition as claimed in claim 1, wherein said composition is in the form of a crosslinked hydrogel or gel.

6. The composition as claimed in claim 1, wherein said composition also contains aminolevulinic acid.

7. The composition as claimed in claim 6, wherein said composition further comprises a combination of chlorophyllin and aminolevulinic acid.

8. The composition as claimed in any one of the preceding claims, characterized in that it also contains diosgenin, or a diosgenin ester.

9. The composition as claimed in claim 1, wherein said composition also contains an alpha-hydroxy or beta-hydroxy acid.

10. The composition as claimed in claim 9, wherein the alpha-hydroxy acid is glycolic acid and the beta-hydroxy acid is salicylic acid.

11. The composition as claimed in claim 1, wherein said composition also contains a depigmenting or lightening agent and/or an antioxidant.

12. A method for cosmetic treatment of the skin, said method comprising the steps of:
applying, to the skin, a composition as claimed in claim 1, and
illuminating said composition by means of a light source of wavelength between 610 and 650 nm.

13. The method for cosmetic treatment of the skin as claimed in claim 12, wherein the light source has a wavelength between 625 and 635 nm.

14. A method for photodynamic therapy for preventing and treating skin aging signs, psoriasis, acne, vitiligo and melasma, and rejuvenating the skin, said method comprising the steps of:
preparing a medicament of chlorophyll, of chlorophyllin or of a derivative thereof represented by general formula (I) of claim 1; and
applying said medicament.
